(19) United States
(12) Patent Application Publication
(10) Pub. No.: US 2023/0263788 A1
(43) Pub. Date: Aug. 24, 2023

(54) COMPANION DIAGNOSTIC FOR AXITINIB

(71) Applicant: The Regents of The University of Michigan, Ann Arbor, MI (US)

(72) Inventors: Paul Swiecicki, Ann Arbor, MI (US); J. Chad Brenner, Ann Arbor, MI (US)

(21) Appl. No.: 18/041,058

(22) PCT Filed: Aug. 10, 2021
(86) PCT No.: PCT/US2021/045372

§ 371 Date: Feb. 8, 2023

Related U.S. Application Data
(60) Provisional application No. 63/064,675, filed on Aug. 12, 2020.

(51) Int. Cl. A61K 31/4439; C12Q 1/6886; A61P 35/04; A61K 45/06

(52) U.S. Cl. CPC A61K 31/4439; C12Q 1/6886; A61P 35/04; A61K 45/06; C12Q 2600/156; C12Q 2600/106

(57) ABSTRACT

The disclosure provides methods for treating cancer with axitinib by first selecting a subject for treatment that has a mutation in one or more genes involved in the phosphoinositide 3-kinase (PI3K) signaling pathway followed by administering a dose of axitinib to the selected subject.

### CROSS REFERENCE TO RELATED APPLICATION

This application claims priority to U.S. Provisional Application No. 63/064,675, filed Aug. 12, 2020, which is hereby incorporated by reference in its entirety.

### FIELD

The present invention relates to methods of using mutations in phosphoinositide 3-kinase (PI3K) signaling pathway genes as a companion diagnostic for treating cancer patients with axitinib.

### BACKGROUND

Head and Neck Squamous Cell Carcinoma (HNSCC) is the 6th most common cancer with 600,000 new cases worldwide each year and an increasing incidence rate due to the high prevalence of human papilloma virus (HPV)-induced HNSCC¹. In fact, oropharyngeal cancer is one of only four cancers increasing in incidence in the United States². Although the majority of patients with HNSCC are cured with multimodality therapy, a significant proportion develop unresectable recurrent or metastatic HNSCC (R/M HNSCC). Despite the recent development of programmed death-1 (PD-1) inhibitors, response rates remain low due to variability within the immune micro-environment³. The median survival for patients newly diagnosed with R/M HNSCC is approximately 12 months.

With increasing molecular characterization of head and neck squamous cell carcinoma, there has been significant interest in targeted therapy⁵. Vascular endothelial growth factor (VEGF) dysregulation has been identified as a crucial process in R/M HNSCC in not only angiogenesis, but also progression, immunosuppression, and immune tolerance,^{6, 7}. Furthermore, VEGF overexpression is associated with advanced disease and poor prognosis^{8, 9}. Given this central role in advanced disease and tumorigenesis, VEGF inhibition is of significant interest as a candidate for targeted therapy.

Axitinib is a multi-receptor tyrosine kinase inhibitor approved in renal cell carcinoma which inhibits several isoforms of VEGF receptor (VEGFR 1, 2, and 3). Furthermore, it has inhibitory activity against PDGFR and downstream effectors of EGFR, both of which are commonly disrupted and contribute to head and neck tumorigenesis,^{5, 10, 11}. Given this mechanism of action and known molecular alterations in R/M HNSCC, axitinib has been evaluated in a phase II study in patients with heavily pretreated R/M HNSCC. A low response (7%) rate was observed with axitinib as a single agent; however, a significant proportion of patients had stable disease (70%) with radiographic findings consistent with treatment response². Differential manifestations of response have been observed with tyrosine kinase inhibitors (e.g., swelling, cystic attenuation), which have the potential of being incorrectly interpreted as progressive disease depending on the criteria employed.

Thus, there remains a need for methods to more accurately identify cancer patients that will respond to axitinib therapy.

### SUMMARY

The disclosure provides a method for treating a cancer in a subject, which method comprises: (a) determining the presence of a mutation in one or more genes involved in the phosphoinositide 3-kinase (PI3K) signaling pathway in a sample obtained from the subject; and (b) administering a dose of axitinib to the subject, whereby the cancer in the subject is treated.

The disclosure also provides axitinib for use in a method of treating a subject with cancer, wherein the method comprises: (a) determining whether a test sample from the subject comprises a mutation in one or more genes involved in the phosphoinositide 3-kinase (PI3K) signaling pathway; and (b) if the test sample from the subject comprises a mutation in one or more genes involved in the PI3K signaling pathway, administering to the subject an effective amount of axitinib.

### BRIEF DESCRIPTION OF THE DRAWING(S)

FIGS. 1A and 1B are graphs of Kaplan Meier Survival Analysis illustrating the overall survival (FIG. 1A) and progression free survival (FIG. 1B) amongst patients treated with axitinib.

FIGS. 2A and 2B are graphs illustrating the maximum degree of response to axitinib treatment by Choi Criteria amongst evaluable patients (FIG. 2A) as well as those with genomic sequencing results, clustered by mutation status (FIG. 2B).

FIG. 3 is a schematic diagram illustrating alteration status of selected genes of interest amongst evaluable patients with sequencing results.

### DETAILED DESCRIPTION OF THE INVENTION

The present disclosure is predicated, at least in part, on the discovery that alterations in PI3K signaling pathway genes are enriched in R/M HNSCC patients that respond to axitinib, Thus, molecular evaluation of PI3K status in patients with HNSCC, and possibly other cancers, may be used as a companion diagnostic to select patients for axitinib therapy.

### Definitions

To facilitate an understanding of the present technology, a number of terms and phrases are defined below. Additional definitions are set forth throughout the detailed description.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA: see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or a RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “variant” refers to the exhibition of qualities that have a pattern that deviates from what occurs in nature. In some embodiments, a variant may also be a mutant.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

The term “tumor,” as used herein, refers to an abnormal mass of tissue that results when cells divide more than they should or do not die when they should. In the context of the present disclosure, the term tumor may refer to tumor cells and tumor-associated stromal cells. Tumors may be benign and non-cancerous if they do not invade nearby tissue or spread to other parts of the organism. In contrast, the terms “malignant tumor,” “cancer,” and “cancer cells” may be used interchangeably herein to refer to a tumor comprising cells that divide uncontrollably and can invade nearby tissues. Cancer cells also can spread or “metastasize” to other parts of the body through the blood and lymph systems. The terms “primary tumor” or “primary cancer” refer to an original, or first, tumor in the body. The term “metastasis,” as used herein, refers to the process by which cancer spreads from the location at which it first arose as a primary tumor to distant locations in the body. The terms “metastatic cancer” and “metastatic tumor” refer to the cancer or tumor resulting from the spread of a primary tumor. It will be appreciated that cancer cells of a primary tumor can metastasize through the blood or lymph systems.

An agent is “cytotoxic” and induces “cytotoxicity” if the agent kills or inhibits the growth of cells, particularly cancer cells. In some embodiments, for example, cytotoxicity includes preventing cancer cell division and growth, as well as reducing the size of a tumor or cancer. Cytotoxicity of tumor cells may be measured using any suitable cell viability assay known in the art, such as, for example, assays which measure cell lysis, cell membrane leakage, and apoptosis. For example, methods including but not limited to trypan blue assays, propidium iodide assays, lactate dehydrogenase (LDH) assays, tetrazolium reduction assays, resazurin reduction assays, protease marker assays, 5-bromo-2′-deoxy-uridine (BrdU) assays, and ATP detection may be used. Cell viability assay systems that are commercially available also may be used and include, for example, CELLTITER-GLO® 2.0 (Promega, Madison, Wis.), VIVAFIX™ 583/603 Cell Viability Assay (Bio-Rad, Hercules, Calif.); and CYTOTOX-FLUOR™ Cytotoxicity Assay (Promega, Madison, Wis.).

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition within the scope and meaning herein.

As used herein, the terms “treatment,” “treating,” and the like, refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

### PI3K Status as Companion Diagnostic

As discussed above, head and neck squamous cell carcinoma patients with mutations in genes involved in the phosphoinositide 3-kinase (PI3K) signaling pathway have been shown to respond better to axitinib treatment than HNSCC patients without such mutations. Thus, mutations in PI3K pathway genes serve as biomarkers for response to axitinib treatment, and the methods described herein may be employed as a companion diagnostic to select cancer patients for axitinib therapy. The U.S. Food and Drug Administration (FDA) defines a “companion diagnostic” as a medical device, often an in vitro device, which provides information that is essential for the safe and effective use of a corresponding drug or biological product. Furthermore, the FDA specifies four areas where a companion diagnostic assay could be essential. (i) to identify patients who are most likely to benefit from a particular therapeutic product; (ii) to identify patients likely to be at increased risk for serious side effects as a result of treatment with a particular therapeutic product; (iii) to monitor response to treatment with a particular therapeutic product for the purpose of adjusting treatment to achieve improved safety or effectiveness, and (iv) to identify patients in the population for whom the therapeutic product has been adequately studied, and found safe and effective, i.e., there is insufficient information about the safety and effectiveness of the therapeutic product in any other population (US FDA. Guidance for Industry and Food and Drug Administration Staff. In Vitro Companion Diagnostic Devices. Aug. 6, 2014; Jørgensen, J. T. and M. Hersom, Ann Transl Med., 4(24): 482 (2016); and Agarwal et al., Pharmgenomics Pers Med., 8: 99-110 (2015). Currently, the term “companion diagnostic” is understood in the art as also encompassing a diagnostic test or biomarker used in a specific context that provides biological and/or clinical information that enables better decision making about the development and use of a potential drug therapy (Austin, M J F. Companion Diagnostics: Reality Check. Cambridge Healthtech Institute (CHI) Next Generation Dx Summit. Pre-Conference Symposium, Aug. 9, 2009. Washington D.C., USA; and Frueh, F. Reality Check on Companion Diagnostics. Cambridge Healthtech Institute (CHI) Next Generation Dx Summit. Pre-Conference Symposium, Aug. 9, 2009. Washington D.C., USA).

The disclosure provides a method for treating a cancer in a subject, which comprises determining the presence of a mutation in one or more genes involved in the phosphoinositide 3-kinase (PI3K) signaling pathway in a sample obtained from the subject. The PI3K (also referred to in the art as “PI3K/AKT” and “(PI3K)/AKT/mammalian target of rapamycin (mTOR)”) signaling pathway is a key regulator of normal cellular processes involved in cell growth, proliferation, metabolism, motility, survival, and apoptosis (Katso et al., Annu Rev Cell Dev Biol., 17: 615-75 (2001); and Engelman et al., Nat Rev Genet., 7: 606-19 (2006)). Aberrant activation of the PI3K pathway promotes the survival and proliferation of tumor cells in many human cancers, as well as resistance to anticancer therapies (Porta et al., Front Oncol., 4: 64 (2014); Huang et al., J Formos Med Assoc., 108: 180-194 (2009); and Martini et al., Ann Med., 46: 372-83 (2014)). PI3K, AKT, a serine/threonine protein kinase also known as protein kinase B (PKB), and mTOR are three major proteins in the pathway. These proteins are typically activated by upstream signaling of tyrosine kinases and other receptor molecules such as hormones and mitogenic factors (Ruggero et al., Oncogene, 24: 7426-7434 (2005)).

The inventive method encompasses determining the presence of at least one mutation in any gene encoding a protein that is involved in the PI3K signaling pathway. Exemplary genes include, but are not limited to, XIAP/BIRC4 (X-linked inhibitor of apoptosis; NM_001167.2); AKT1 (v-akt murine thymoma viral oncogene homolog 1; NM_005163); TWIST1 (Twist homolog 1 (Drosophila); NM_000474.3); BAD (BCL2-associated agonist of cell death NM_004322.2); CDKN1A/p21 (Cyclin-dependent kinase inhibitor 1A (p21, Cip1); NM_000389.2); ABL1 (v-abl Abelson murine leukemia viral oncogene homolog 1; NM_005157.3); CDH1 (Cadherin 1, type 1, E-cadherin; NM_004360.3); TP53 (Tumor protein p53; NM_000546); CASP3 (Caspase 3, apoptosis-related cysteine peptidase; NM_004346.2); PAK1 (p21/Cdc42/Rac1-activated kinase 1; NM_002576.4); GAPDH (Glyceraldehyde-3-phosphate dehydrogenase; NM_002046.3); PIK3CA (Phosphoinositide-3-kinase, catalytic, a-polypeptide; NM_006218.2); FAS (TNF receptor superfamily, member 6; NM_000043.3); AKT2 (v-akt murine thymoma viral oncogene homolog 2; NM_001626.3); FRAP1/mTOR (FK506 binding protein 12-rapamycin associated protein 1; NM_004958.3); FOXO1A (Forkhead box O1; NM_002015.3); PTK2 (FAK) (PTK2 protein tyrosine kinase 2; NM_005607.3); CASP9 (Caspase 9, apoptosis-related cysteine peptidase; NM_001229.2); PTEN (Phosphatase and tensin homolog; NM_000314.4); CCND1 (Cyclin D1; NM_053056.2); NFKB1 (Nuclear factor k-light polypeptide gene enhancer B-cells 1; NM_003998.2); GSK3B (Glycogen synthase kinase 3-b; NM_002093.2); MDM2 (Mdm2 p53 binding protein homolog (mouse); NM_002392.2); and CDKNIB (Cyclin-dependent kinase inhibitor 1B (p27, Kip1); NM_004064.3) (see, e.g., Catasus et al., Modern Pathology, 23: 694-702 (2010); Vivanco I, Sawyers C L., Nat Rev Cancer, 2: 489-501 (2002); Cully et al., Nat Rev Cancer, 6: 184-192 (2006); Bader et al., Nat Rev Cancer, 5: 921-929 (2005); and Samuels Y, Ericson K., Curr Opin Oncol, 18: 77-82 (2006)). In some embodiments, the method involves determining the presence of at least one mutation in one or more genes selected from PTEN, PIK3CA, and AKT.

The term “mutation,” as used herein, encompasses any structural change made to a wild-type nucleic acid sequence. The one or more PI3K pathway genes may have any type of mutation, and the mutation may or may not result in a protein with altered function. In some embodiments, however, the one or more mutations impairs the function of protein encoded by the PI3K pathway gene. For example, the mutation may be a missense mutation, a nonsense mutation, deletion or insertion of one or more nucleotides, duplication, amplification, a frameshift mutation, repeat expansion, and/or other modifications that affect the structural integrity or nucleotide sequence. A “missense mutation” is a change in one DNA base pair that results in the substitution of one amino acid for another in the encoded protein. A “nonsense mutation” is a change in one DNA base pair that converts a sense codon to a chain-terminating codon, resulting in the translation of an abnormally short polypeptide generally with altered functionality. A “duplication” comprises a piece of DNA that is abnormally copied one or more times. “Amplification” is a mutation that increases the copy number of a specific DNA segment in a cell. A “frameshift mutation” occurs when the addition or loss of DNA bases changes a gene's reading frame. Insertions, deletions, and duplications can all induce frameshift mutations. “Repeat expansion” refers to a mutation that increases the number of times that a short (e.g., 3 or 4 base pairs) DNA sequence present in the gene is repeated.

The presence of a mutation in one or more genes involved in the PI3K signaling pathway may be determined using any suitable method, a variety of which are known in the art. Such methods include, for example, restriction fragment length polymorphism (RFLP) analysis, Sanger sequencing, high-throughput sequencing (also referred to as “next generation sequencing”), tracking of indels by decomposition (TIDE) software, T7 endonuclease 1 (T7E1) assay, PCR based methods (e.g., RT-PCR, real-time or quantitative PCR, multiplex PCR, and nested PCR), multiplex ligation-dependent probe amplification (MLPA), denaturing gradient gel electrophoresis (DGGE), single strand conformational polymorphism (SSCP), chemical cleavage of mismatch (CCM), protein truncation test (PTT), and oligonucleotide ligation assay (OLA) (see, e.g., Mahdieh, N. and B. Rabbani, Iran J. Pediatr., 23(4): 375-388 (2013); Al-Haggar, M., Gene Technology 2: e104 (2013). doi: 10.4172/2329-6682.1000e104; and Frayling et al., PCR-Based Methods for Mutation Detection. In: Coleman W. B., Tsongalis G. J. (eds) Molecular Diagnostics. Humana Press (2006)).

### Sample

The terms “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), mucosal biopsy tissue and brushed cells, sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the foregoing. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. In some embodiments, the biological sample may comprise tumor tissue, suspected tumor tissue, or lymph node tissue. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. It will be appreciated that obtaining a biological sample from a subject may comprise extracting the biological sample directly from the subject or receiving the biological sample from a third party.

### Treatment of Cancer

The disclosure provides a method of treating cancer. Ideally, administration of axitinib as described herein inhibits the growth of cancer cells from a primary tumor or cancer or a metastatic tumor or cancer. In some embodiments, the method induces cytotoxicity in tumor cells or cancer cells.

A cancer or tumor may arise in any organ or tissue. For example, the cancer or tumor may be a carcinoma (cancer arising from epithelial cells), a sarcoma (cancer arising from bone and soft tissues), a lymphoma (cancer arising from lymphocytes), a melanoma, or brain and spinal cord tumors. The tumor or cancer cells can arise in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid). More particularly, tumors or cancers of the digestive system can arise in the esophagus, stomach, small intestine, colon, rectum, anus, liver, gall bladder, and pancreas. Cancers or tumors of the respiratory system can arise in the larynx, lung, and bronchus and include, for example, non-small cell lung carcinoma. Cancers or tumors of the reproductive system can affect the uterine cervix, uterine corpus, ovaries, vulva, vagina, prostate, testis, and penis. Cancers of the urinary system can arise in the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, etc.). The cancer may be a primary tumor, or alternatively, or a metastatic tumor. In some embodiments, the cancer is a head and neck cancer, such as a squamous cell head and neck carcinoma (HNSCC) or unresectable recurrent or metastatic head and neck squamous cell carcinoma (R/M HNSCC).

Targeted therapy has demonstrated promise in pre-clinical studies in HNSCC. Alterations in PI3KCA, CDKN2A, and EGFR suggest head and neck cancer is a candidate for the development of targeted therapeutics. Tyrosine kinase inhibitors offer the benefit of targeting numerous pathways (i.e., VEGFR, EGFR, PDGFR) and isoforms simultaneously. Evidence suggests that VEGF inhibition is immunomodulatory via numerous mechanisms, including production of IFNγ, reversal of the immunosuppressive microenvironment, and augmented activity of CD8+ T cells via hypoxia-inducible factor-la secondary to tumor hypoxia¹⁹⁻²¹. As VEGFR inhibition may prime the immune system for response to immunotherapy, sequential use may be a modality to decrease toxicities yet still gain therapeutic synergy.

Thus, once a subject is determined to have at least one mutation in one or more genes involved in the PI3K signaling pathway, the method comprises administering a dose of axitinib to the subject, whereby the cancer in the subject is treated. Axitinib (marketed in the U.S. as INLYTA® by Pfizer, Inc.) is approved in the U.S. for the first-line treatment of advanced renal cell carcinoma (RCC) in combination with avelumab, for the first-line treatment of advanced RCC in combination with pembrolizumab, and as a single agent for the treatment of advanced renal cell carcinoma (RCC) after failure of one prior systemic therapy. As discussed above, axitinib is a small molecule receptor tyrosine kinase inhibitor having the following structure:

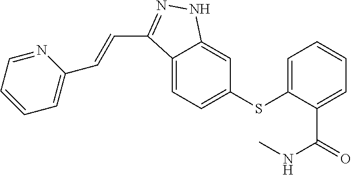 [SMILES: CNC(=O)c1ccccc1Sc1ccc2c(/C=C/c3ccccn3)n[nH]c2c1]

Axitinib is active against several isoforms of VEGF receptor (VEGFR 1, 2, and 3) and exhibits inhibitory activity against PDGFR, c-Kit, and downstream effectors of EGFR.

Any suitable dose of axitinib may be administered to the subject, so long as axitinib is efficiently delivered to target cancer cells such that cancer cell growth is inhibited. To this end, the inventive method comprises administering a “therapeutically effective amount” of axitinib. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of axitinib to elicit a desired response in the individual. For example, a therapeutically effective amount of axitinib is an amount which is cytotoxic to cancer cells, such that the cancer or tumor is eliminated.

Axitinib is currently available in the U.S. in 1 mg and 5 mg oral dosage forms (tablets). Thus, in some embodiments, a starting dose of 5 mg axitinib may be administered twice daily to the subject. It will be appreciated that dose adjustments can be made based on individual safety and tolerability. For example, patients who tolerate axitinib for at least two consecutive weeks with no adverse reactions Grade>2 (according to the Common Toxicity Criteria for Adverse Events [CTCAE]), are normotensive, and are not receiving anti-hypertension medication, may have their dose increased from 5 mg to 7 mg twice daily, or up to 10 mg twice daily using the same criteria. Adverse reactions may be managed by temporarily reducing the dose of axitinib, such as, for example, to 3 mg or 2 mg twice daily.

Axitinib may be formulated for administration to a mammal, particularly a human, using standard administration techniques and routes. Suitable administration routes include, but are not limited to, oral, intravenous, intraperitoneal, subcutaneous, subcutaneous, intramuscular, or parenteral administration. The term “parenteral,” as used herein, includes intravenous, intramuscular, subcutaneous, rectal, vaginal, and intraperitoneal administration. In other embodiments, an axitinib formulation may be administered to a mammal using systemic delivery by intravenous, intramuscular, intraperitoneal, or subcutaneous injection. Ideally, axitinib is formulated for oral administration (e.g., as a tablet or capsule).

In some embodiments, the subject has received at least one cancer treatment prior to the administration of the dose of axitinib. The subject may have previously received any cancer treatment known in the art, such as, for example, surgery, chemotherapy, radiation therapy, or cancer immunotherapy, hormone therapy, and/or stem cell transplantation. Chemotherapeutic agents include, for example, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, anti-vascular endothelial growth factor compounds (“anti-VEGFs”), anti-epidermal growth factor receptor compounds (“anti-EGFRs”), 5-fluorouracil, and the like. In other embodiments, the subject has been treated with an immune checkpoint inhibitor (discussed further below) prior to the administration of the dose of axitinib. For example, the subject may have received treatment with a PD-1 inhibitor prior to administration of the dose of axitinb.

In some embodiments, the disclosed method promotes inhibition of cancer cell proliferation, the eradication of cancer cells, and/or a reduction in the size of at least one cancer or tumor such that the cancer or tumor is treated in a mammal (e.g., a human). By “treatment of cancer” is meant alleviation of a cancer in whole or in part. In one embodiment, the disclosed method reduces the size of a cancer or tumor by at least about 20% (e.g., cancer about 25%, about 30%, about 35%, about 40%, about 45%, about 50%, about 55%, about 60%, about 65%, about 70%, about 75%, about 80%, about 85%, about 90%, or about 95%). Ideally, the cancer or tumor is completely eliminated.

In some embodiments, the disclosed method may further comprise administering a cancer immunotherapeutic to the subject simultaneously with or subsequently to administration of the dose of axitinib. A “cancer immunotherapeutic” is any agent, substance, compound, or method used to treat cancer that involves or uses components of a patient's immune system. In some embodiments, cancer immunotherapeutics may include antibodies that bind to, and inhibit the function of, proteins expressed by cancer cells. Other cancer immunotherapies include vaccines and T cell infusions. Thus, the cancer immunotherapeutic used herein may include, for example, immune checkpoint inhibitors, monoclonal antibodies, cancer vaccines, immune system modulators, and/or T-cell transfer therapy. Cancer immunotherapy is further described in, e.g., Finck et al., Nat Commun, 11: 3325 (2020). doi.org/10.1038/s41467-020-17140-5; and Karp et al. (eds.), Handbook of Targeted Cancer Therapy v and Immunotherapy, Second Edition, Lippincott, Williams & Wilkins, 408 pp. (2018)).

In some embodiments, the method further comprises administering to the subject an immune checkpoint regulator. Immune checkpoints are molecules on immune cells that must be activated or inhibited to stimulate immune system activity. Tumors can use such checkpoints to evade attacks by the immune system. The immune checkpoint regulator may be an antagonist of an inhibitory signal of an immune cell, also referred to as a “checkpoint inhibitor,” which blocks inhibitory checkpoints (i.e., molecules that normally inhibit immune responses). For example, the immune checkpoint regulator may be an antagonist of A2AR, BTLA, B7-H3, B7-H4, CTLA4, GAL9, IDO, KIR, LAG3, PD-1, TDO, TIGIT, TIM3 and/or VISTA. Checkpoint inhibitor therapy therefore can block inhibitory checkpoints, restoring immune system function. Currently approved checkpoint inhibitors target the molecules CTLA4, PD-1, and PD-L 1, and include ipilimumab (YERVOY®), nivolumab (OPDIVO®), pembrolizumab (KEYTRUDA®), atezolizumab (TECENTRIQ®), avelumab (BAVENCIO®), and durvalumab (IMFINZI®). Any suitable checkpoint inhibitor, such as those described in, e.g., Kyi, C. and M. A. Postow, Immunotherapy, 8(7): 821-37 (2016); Collin, M., Expert Opin Ther Pat., 26(5): 555-64 (2016); Pardoll, D. M., Nat Rev Cancer, 12(4): 252-6 (2012); and Gubin et al., Nature, 515(7528): 577-81 (2014)) may be used in combination with the disclosed method. In other embodiments, the immune checkpoint regulator may be an agonist of an immune cell stimulatory receptor, such as an agonist of BAFFR, BCMA, CD27, CD28, CD40, CD122, CD137, CD226, CRTAM, GITR, HVEM, ICOS, DR3, LTBR, TACI and/or OX40.

The following example further illustrates the invention but, of course, should not be construed as in any way limiting its scope.

### Example

This example demonstrates the efficacy of axitinib in metastatic head and neck cancer using novel radiographic response criteria.

A previous phase II study evaluated axitinib in patients with heavily pretreated R/M HNSCC (see Swiecicki et al., Invest New Drugs 33: 1248-56 (2015)). This work demonstrated a low response (7%) rate with single agent axitinib; however, a significant proportion of patients had stable disease (70%) with radiographic findings consistent with treatment response. Moreover, the population had an impressive overall survival (10.9 months) suggesting that efficacy was perhaps was not captured. Thus, it was hypothesized that axitinib induced significant anti-tumor activity in R/M HNSCC, but the Response Evaluation Criteria in Solid Tumors (RECIST) used failed to appropriately capture responders and may have inappropriately suggested tumor progression. Indeed, differential manifestations of response have been seen with the use of tyrosine kinase inhibitors (e.g., swelling, cystic attenuation), which have the potential of being improperly interpreted as progressive disease by RECIST. Such observations prompted the development of the Choi Criteria (Choi et al., J Clin Oncol, 25: 1753-9, 2007), which takes into account tumor size and drug-induced necrosis.

Based on these findings, another phase II study was conducted to investigate the clinical activity of axitinib in R/M HNSCC using the Choi Criteria for response assessment. The hypothesis was that axitinib would have significant anti-tumor activity as judged by the Choi Criteria and result in an improvement in the six-month overall survival compared to a historical control.

### Materials and Methods

### Patient Eligibility

The phase 2 open label trial was approved by the Institutional Review Board (IRBMED) of the University of Michigan Rogel Cancer Center (NCT02762513). All patients provided written informed consent. Patients≥18 years old with histologically documented unresectable recurrent or metastatic head and neck squamous cell carcinoma were eligible. All patients were required to have the presence of measurable disease by CT scan or cutaneous lesions≥10 mm not assessable on imaging but present on physical exam, ECOG performance status of 0-2, and life expectancy of ≥12 weeks. Adequate hematopoietic, hepatic, and renal function were required and defined as: absolute neutrophil count≥1.5×10⁹ cell/ml, platelets≥75,000 cells/mm³, hemoglobin≥9.0 g/dL, concentrations of total serum bilirubin within 1.5× the upper limit of normal (ULN), aspartate aminotransferase (AST) and alanine aminotransferase (ALT) within 2.5× institutional upper limits of normal unless there were liver metastases, in which case AST and ALT within 5.0×ULN, serum creatinine clearance≥30 ml/min, urinary protein<2+). Women of childbearing potential must have had a negative serum or urine pregnancy test within 3 days prior to treatment.

Patients with tumors encasing major blood vessels, active hemoptysis (>½ teaspoon of bright red blood per day), or currently using therapeutic anticoagulation were excluded as were those with gastrointestinal abnormalities resulting in impaired absorption. Treatment with epidermal growth factor receptor inhibitors within 30 days preceding study entrance was prohibited. Patients were excluded if they had uncontrolled hypertension prior to enrollment which was defined as a systolic blood pressure readings>140 mm Hg and/or a diastolic blood pressure readings>90 mm Hg.

### Treatment Plan

Enrolled patients underwent a complete history and physical examination, baseline laboratory studies (CBC with differential, comprehensive metabolic profile, TSH, urinalysis) and radiographic staging studies (CT Neck/Chest and others as clinically warranted). If cutaneous lesions were not assessable for response by imaging, pictures of the target lesion(s) were obtained as well. All screening assessments were completed within 28 days prior to the start of treatment.

Patients were initiated on axitinib at 5 mg twice daily with a cycle length of 28 days. Dose escalation was planned at 2 weeks (to 7 mg twice daily) and 3 weeks to the goal of 10 mg twice daily in the absence of grade 2 or greater toxicities. Patients were seen for toxicity assessment and laboratory assessments (CBC, CMP, TSH, UA) at two weeks, four weeks, and then monthly after treatment initiation. Dose escalation could be resumed at the next visit if toxicities diminished to grade 1 or less. Treatment was continued until disease progression, unacceptable toxicity, patient withdrawal of consent, or investigator discretion.

### Evaluation of Response

Response assessment was performed after two cycles of axitinib treatment and continued every two cycles. Radiographic assessments obtained at enrollment were obtained at each time point. Similarly, if a physical exam was being used for response assessment of cutaneous lesions, pictures were taken at each time point. Photographs as well as imaging studies were submitted to the University of Michigan Tumor Response and Assessment Core. Radiologic response was determined according to the Choi Criteria¹³.

### Statistical Considerations

This expansion study was designed based on the previous study supporting an improvement in survival in patients with R/M HNSCC treated with axitinib. Although consideration was given to adjusting this original study to a Bayesian expansion trial design, it was ultimately decided to begin a new cohort to test for an improvement in survival under the same assumptions of mortality rate as the previous study. Of note, treatment continuation decisions for this trial were based on Choi criteria that, as previously reported¹², differed considerably from RECIST decisions when evaluated in the original trial.

The primary aim was to compare 6-month overall survival after treatment with axitinib in patients with unresectable, recurrent or metastatic head and neck cancer to historical rates. Based on results in the literature, we assumed a 6-month mortality rate of 50% under current standard care in this patient population¹⁴. A sample size of 37 patients was planned to test whether survival after treatment with axitinib is improved to 70% at 6-months compared to 50% with an upper tailed test of binomial proportion. No interim analyses for activity were planned. Based on observed clinical benefit and slowed accrual rate, an unplanned interim analysis was performed after enrollment of 29 patients. Data was analyzed by the study statistician; a statistically meaningful improvement in survival was identified in this analysis and the decision was made to close to further accrual.

Overall survival (OS) was defined as the time from study enrollment to death from any cause. Six month overall survival was the proportion of patients who received at least one cycle of axitinib alive 6 months after study enrollment and 95% confidence intervals were estimated using the Wilson score interval method. Treatment-related adverse events were graded according to the Common Terminology for Adverse Events version 4.03. Response rate was defined as the sum of patients with complete response (CR) and partial response (PR) per the Choi Criteria. Statistical analysis was performed with SAS v14.3 software (Carey, N.C.).

### Results

### Patient Characteristics

Twenty nine patients were enrolled, one of which died prior to treatment with axitinib. All twenty eight patients who received at least one dose of axitinib were included for toxicity analysis of which the baseline characteristics are summarized in Table 1. The mean age was 63.9 years old (range: 37-80) and the majority of patients (61%, n=17) had an ECOG performance score of 1, indicating mild impairment. The primary site of disease for most patients was the oropharynx (42.6%, n=13) and the majority of study participants were HPV negative (60.7%, n=17). The majority of patients (61%, n=17) had at least one previous systemic treatment in the metastatic setting with the number or previous lines of treatments ranging from 0-5. Seventeen patients (61%) were refractory to platinum therapy defined as progression within 180 days of chemotherapy and twelve patients (43%) were previously treated with a PD-1 inhibitor.

TABLE 1
Patient Demographics and Clinical Characteristics
This table describes the baseline demographics of the
patients included in analysis for efficacy.

| Age | n | 28 | |
|---|---|---|---|
| | Mean | 63.9 | |
| | Median (range) | 64.5 | (37-80) |
| Gender, n (%) | Male | 25 | (89%) |
| | Female | 3 | (11%) |
| ECOG Performance Status, | 0 (Fully functional) | 11 | (39%) |
| n (%) | 1 (Minor Impairment) | 17 | (61%) |
| Disease Primary Site, n (%) | Oral Cavity | 2 | (7.1%) |
| | Oropharynx | 13 | (46%) |
| | Larynx | 4 | (13.3%) |
| | Nasopharynx | 3 | (10.7%) |
| | Cutaneous | 6 | (21.4%) |
| HPV Status, n (%) | Positive | 10 | (35.7%) |
| | Negative | 17 | (60.7%) |
| | Unknown | 1 | (3.6%) |
| Previous Lines of Therapy | 0 | 11 | (39%) |
| | 1 | 6 | (21.4%) |
| | 2 | 5 | (17.8%) |
| | 3+ | 6 | (21.4%) |
| Previous Exposure to | Sensitive | 11 | (39.2%) |
| Platinum | Refractory | 17 | (60.7%) |
| Previous Exposure to | PR | 3 | (25%) |
| PD-1 Inhibitor | SD | 3 | (25%) |
| | PD | 6 | (50%) |

### Toxicity

The median duration of treatment was 3 cycles (range: 1-9). The most common toxicities included fatigue (75%), hypertension (54%), nausea (32%), and diarrhea (25%) (Table 2). Bleeding was observed in 5 patients, including one patient with a grade 3 lower GI bleed, all of which spontaneously resolved and did recur with re-initiation of axitinib. Grade 3 or 4 severe toxicities were seen in 16 patients (57%). Severe toxicities included fatigue (21%), hypertension (7%), and mucositis (7%). No grade 5 events were reported. Overall, observed toxicities were consistent with that previously reported in the literature^{15, 16}.

TABLE 2
Treatment Related Toxicities
This table demonstrates the toxicities observed in the entire
study population (n = 29) with a frequency of greater than 10%

| | | | | All |
|---|---|---|---|---|
| Toxicity | Grade 1 or 2 | | Grade 3 or 4 | Grades |
| Fatigue | 15 | (54%) | 6 (21%) | 21 (75%) |
| Hypertension | 13 | (46%) | 2 (7%) | 15 (54%) |
| Oral Mucositis | 2 | (7%) | 2 (7%) | 4 (14%) |
| Diarrhea | 6 | (21%) | 1 (4%) | 7 (25%) |
| Oral pain | 2 | (7%) | 1 (4%) | 3 (11%) |
| Bleeding | 4 | (14%) | 1 (4%) | 5 (18%) |
| Nausea | 9 | (32%) | 0 (0%) | 9 (32%) |
| Weight loss | 7 | (25%) | 0 (0%) | 7 (25%) |
| Anorexia | 6 | (21%) | 0 (0%) | 6 (21%) |
| Aspartate | 6 | (21%) | 0 (0%) | 6 (21%) |
| aminotransferase | | | | |
| increased | | | | |
| Dysgeusia | 5 | (18%) | 0 (0% ) | 5 (18%) |
| Vomiting | 5 | (18%) | 0 (0%) | 5 (18%) |
| Hoarseness | 4 | (14%) | 0 (0%) | 4 (14%) |
| Sore throat | 4 | (14%) | 0 (0%) | 4 (14%) |
| Dehydration | 3 | (11%) | 0 (0%) | 3 (11%) |

### Efficacy

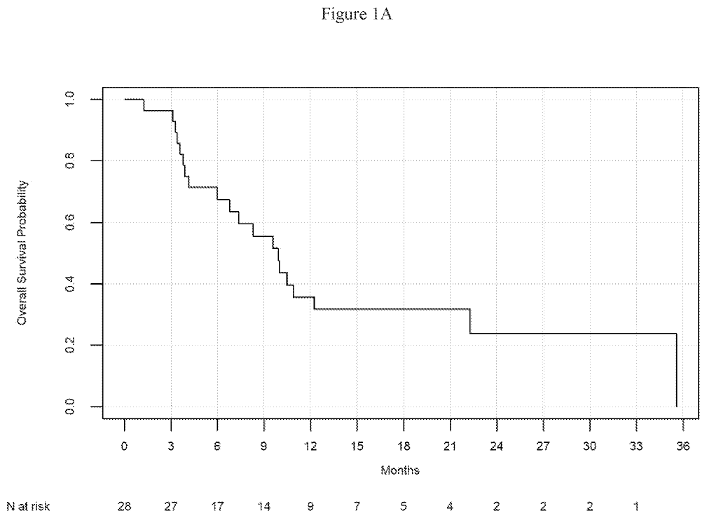

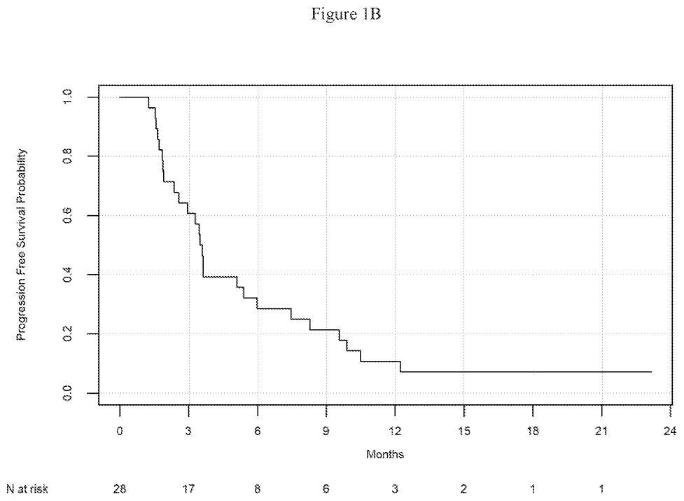

The median follow up duration among the twenty eight study participants was 18 (range- 1-36) months and no patients remain on therapy. Follow-up on patients still alive ranges from 5 to 32 months. The 6 month overall survival was 71% (95% CI: 53-85%) (Table 3). This met the protocol defined criteria for supporting evidence of clinical benefit. The median progression free survival was 3.5 months (95% CI: 2.4-5.4 months) and median overall survival was 9.8 months (95% CI: 5.9-12.2 months) (FIG. 1).

TABLE 3
Treatment Efficacy
This table describes the efficacy and outcomes among
A) evaluable patients and b) those with sequencing results
A

| 6 month PFS (95% CI)¹ | 32% | (18%, 51%) |
|---|---|---|
| Median PFS, days (95% CI)¹ | 107.5 | (72-164) |
| | 3.5 months | |
| 6 month OS (95% CI)¹ | 71% | (53%, 85%) |
| Median OS, days KM estimate (95% CI)¹ | 301 | (182, 372) |
| | 9.8 months | |
| Best Overall Response Rate | 42% | |
| Progressive Disease (PD), n (%) | 10 | (36%) |
| Stable Disease (SD), n (%) | 3 | (11%) |
| Partial Response (PR), n (%) | 11 | (39%) |
| Complete Response (CR), n(%) | 1 | (3%) |
| Off Treatment before 8 week scan, n (%) | 3 | (11%) |

¹6 month survival proportion and 95% confidence interval estimated using Wilson score interval method.

B

| Response Rate (# responders/patients) | | |
|---|---|---|
| | Mutant | Wild-Type |

| | | | | |
|---|---|---|---|---|
| PI3K Signaling Pathway Alterations | 75% | (6/8) | 17% | (2/12) |
| Non-Cutaneous Squamous Cell Carcinoma | 86% | (6/7)^{A} | 12% | (1/8) |
| Cutaneous Squamous Cell Carcinoma | 0% | (0/1) | 25% | (1/4)^{B} |
| KMT2C/D Mutations | 33% | (2/6) | 50% | (6/12) |
| Non-Cutaneous Squamous Cell Carcinoma | 66% | (2/3)^{C} | 50% | (5/10) |
| Cutaneous Squamous Cell Carcinoma | 0% | (0/3) | 50% | (1/2)^{B} |

^{A}Remaining patient had SD as best response to therapy.
^{B}Patient had a KDR (VEFGR2) S110F mutation and exhibited a complete response
^{C}Both patients who exhibited a response had synchronous mutations in the PI3K signaling pathway

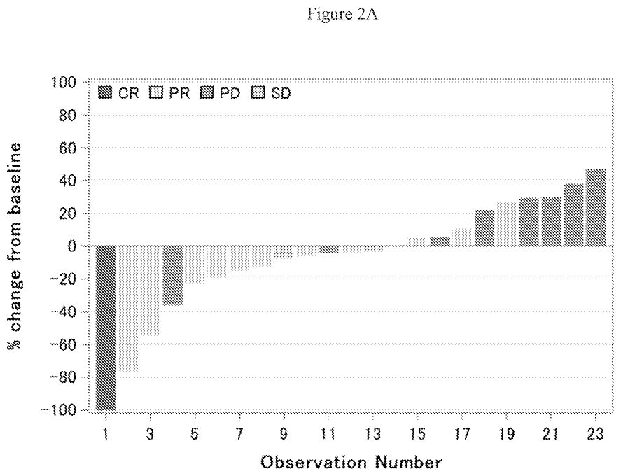

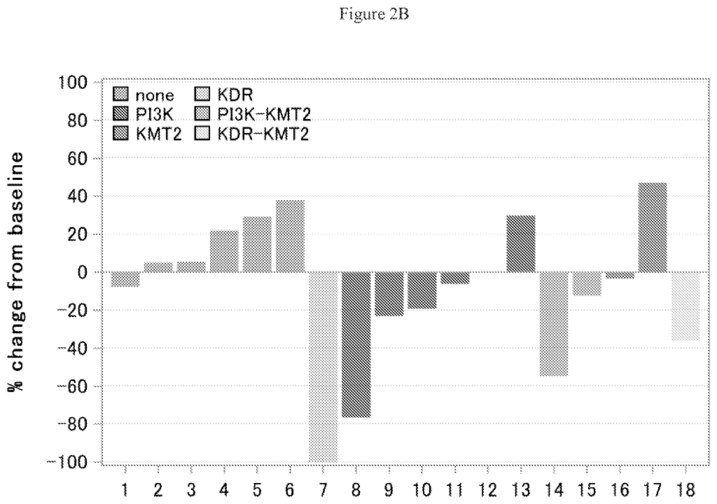

Three patients completed trial participation prior to response imaging; one due to adverse effects but was clinically noted to have progressive disease, one of whom died due to progressive disease, and a third whom withdrew from the study. The overall response rate was 42% and a disease control rate of 53%. The waterfall plot in FIG. 2A graphically demonstrates the depth of response amongst participants evaluable for response. Only one patient with cutaneous squamous cell carcinoma demonstrated a response to therapy. This patient had a mutation in KDR (VEGFR2) and achieved a durable complete response. All of the remaining six patients with cutaneous squamous cell carcinoma had progressive disease.

One patient had a durable complete response as shown. Eleven patients were subsequently treated with a PD-1 inhibitor. Response assessment demonstrated a complete response in one patient, partial response in four patients, stable disease in one patient, and progressive disease was seen in the remaining five.

### Correlative Studies

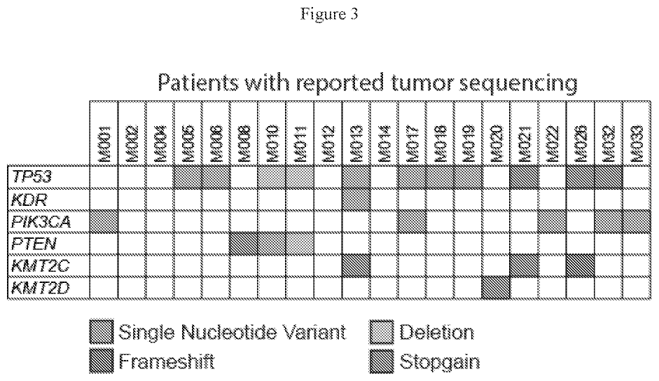

To evaluate the association between genomic alterations, tumor characteristics, and clinical outcomes, results were analyzed from patients who had commercial next generation sequencing previously performed (n=20). The investigators defined a set of genes (sequenced as part of all NGS panels) and recurrent alterations are shown in FIG. 3. Importantly, while no mutations were identified in FLT1 (VEGFR1), FLT4 (VEGFR3), PDGFR or KIT, two patients had mutations in KDR (VEGFR2), including a S1100F mutation as well as a patient with two mutant alleles R1032Q and G638R. The ability of axitinib to inhibit these mutant forms of KDR is unknown; however, the patient with the S110F mutation had a complete response while the other had progressive disease. Importantly, 55% (11/20) of the patients had TP53 alterations, 40% (8/20) of the patients harbored alterations to genes in the PI3K pathway, including PTEN and PIK3CA, and, 30% (6/20) of the patients had mutations in either KMT2C (MLL2) or KMT2D (MLL3).

The degree of response and pathway alterations were correlated for exploratory analysis (FIG. 2B). The relative response rate for patients with mutations in the PI3K pathway was 75%, as compared to 39% in patients lacking PI3K pathway mutations (6/8 versus 2/12 patients). In terms of the KMT2C/D pathway, the response rate was 33% in the mutant population versus 50% in the rest of the population (2/6 versus 6/12 patients). Given the differential responses seen between patients with cutaneous squamous cell carcinoma versus non-cutaneous primaries, the response rates were further explored, as shown in Table 3. Although sample sizes were limited, mutations in the PI3K pathway were associated with a higher response rate than the wild type population in the non-cutaneous squamous cell carcinoma (86% versus 12%).

### Discussion

In this phase 2 study of patients with heavily pretreated unresectable recurrent or metastatic head and neck squamous cell carcinoma (R/M HNSCC), axitinib demonstrated an improvement in 6 month overall survival compared to a historical controls (70% vs 50%). Furthermore, treatment resulted in significant response rates and lower rates of severe toxicities.

There is increasing recognition of variable radiographic manifestations of response with the advent of novel classes of therapeutics. Most recognized is the ‘pseudoprogression’ observed with immunotherapy which prompted development of iRECIST to capture atypical responses²⁵. The Choi Response Criteria have been best evaluated in gastrointestinal stromal tumors (GISTs) where, compared to RECIST, they have been demonstrated to better predict survival³. The previous trial using RECIST revealed a low RECIST assessed objective response rate but a paradoxically high impressive overall survival rate in heavily pre-treated patients¹². As such, it was hypothesized that treatment responses were underappreciated with the use of RECIST and the Choi Criteria may be more appropriate for discerning patients deriving benefiting from therapy. With the utilization of the Choi Criteria in this study, a response rate of 42% was observed with an additional 11% having stable disease. Furthermore, use of these response criteria for treatment decisions resulted in an improvement in overall survival compared to historical controls, suggesting that that the Choi Criteria appropriately identified treatment responders and that axitinib is an effective therapeutic in heavily pretreated R/M HNSCC.

The above study demonstrates a median overall survival of 9.8 months in a heavily pretreated population of which 61% received greater than one line of systemic therapy, 61% of patients were platinum refractory, and 42% of patients were refractory or PD-1 inhibitors. This result is surprising due to the complex array of genetic alterations observed in advanced HNSCC patients. For example, through the available genomic data in this study, two patients with tumors containing KDR (VEGFR2) mutations. Unfortunately, the functional significance of these alterations are currently unknown.

Importantly, the above study is the first demonstration of a clinical link between PI3K status and response to axitinib. Multiple potential mechanisms may account for the relationship, for example, tumors with PI3K alterations are often induce angiogenesis through VEGF-regulated cytokine mechanisms, and this process may be critical for the survival of PI3K-dependent tumors²⁶.

The results described herein demonstrate that axitinib treatment is associated with improved survival in patients with heavily pre-treated head and neck cancer. The Choi Criteria were able to classify treatment responses amongst patients with an atypical radiographic response. Exploratory analysis suggests that marked response rates are seen with the use of a single agent ICI after axitinib (RR: 45%) and patients with PI3K pathway alterations may derive exceptional benefit from therapy (RR: 75% vs. 17%).

### REFERENCES

All references, including publications, patent applications, and patents, cited herein are hereby incorporated by reference to the same extent as if each reference were individually and specifically indicated to be incorporated by reference and were set forth in its entirety herein.
1. Chaturvedi A K, Engels E A, Pfeiffer R M, et al: Human papillomavirus and rising oropharyngeal cancer incidence in the United States. J Clin Oncol 29:4294-301, 2011
2. Siegel R L, Miller K D, Jemal A: Cancer statistics, 2020. CA Cancer J Clin 70:7-30, 2020
3. Mandal R, Senbabaoglu Y, Desrichard A, et al: The head and neck cancer immune landscape and its immunotherapeutic implications. JCI Insight 1:e89829, 2016
4. Burtness B, Harrington K J, Greil R, et al: Pembrolizumab alone or with chemotherapy versus cetuximab with chemotherapy for recurrent or metastatic squamous cell carcinoma of the head and neck (KEYNOTE-048): a randomised, open-label, phase 3 study. Lancet 394:1915-1928, 2019
5. Cancer Genome Atlas N: Comprehensive genomic characterization of head and neck squamous cell carcinomas. Nature 517:576-82, 2015
6. Strauss L, Volland D, Kunkel M, et al: Dual role of VEGF family members in the pathogenesis of head and neck cancer (HNSCC): possible link between angiogenesis and immune tolerance. Med Sci Monit 11:BR280-92, 2005
7. Hasina R, Whipple M E, Martin L E, et al: Angiogenic heterogeneity in head and neck squamous cell carcinoma: biological and therapeutic implications. Lab Invest 88:342-53, 2008
8. Smith B D, Smith G L, Carter D, et al: Prognostic significance of vascular endothelial growth factor protein levels in oral and oropharyngeal squamous cell carcinoma. J Clin Oncol 18:2046-52, 2000
9. Kyzas P A, Cunha I W, Ioannidis J P: Prognostic significance of vascular endothelial growth factor immunohistochemical expression in head and neck squamous cell carcinoma: a meta-analysis. Clin Cancer Res 11:1434-40, 2005
10. Ongkeko W M, Altuna X, Weisman R A, et al: Expression of protein tyrosine kinases in head and neck squamous cell carcinomas. Am J Clin Pathol 124:71-6, 2005
11. Bran B, Bran G, Hormann K, et al: The platelet-derived growth factor receptor as a target for vascular endothelial growth factor-mediated anti-angiogenetic therapy in head and neck cancer. Int J Oncol 34:255-61, 2009
12. Swiecicki P L, Zhao L, Belile E, et al: A phase II study evaluating axitinib in patients with unresectable, recurrent or metastatic head and neck cancer. Invest New Drugs 33:1248-56, 2015
13. Choi H, Charnsangavej C, Faria S C, et al: Correlation of computed tomography and positron emission tomography in patients with metastatic gastrointestinal stromal tumor treated at a single institution with imatinib mesylate: proposal of new computed tomography response criteria. J Clin Oncol 25:1753-9, 2007
14. Herbst R S, Arquette M, Shin D M, et al: Phase II multicenter study of the epidermal growth factor receptor antibody cetuximab and cisplatin for recurrent and refractory squamous cell carcinoma of the head and neck. J Clin Oncol 23:5578-87, 2005
15. Motzer R J, Escudier B, Tomczak P, et al: Axitinib versus sorafenib as second-line treatment for advanced renal cell carcinoma: overall survival analysis and updated results from a randomised phase 3 trial. Lancet Oncol 14:552-62, 2013
16. Hutson T E, Lesovoy V, Al-Shukri S, et al: Axitinib versus sorafenib as first-line therapy in patients with metastatic renal-cell carcinoma: a randomised open-label phase 3 trial. Lancet Oncol 14:1287-94, 2013
17. Vermorken J B, Trigo J, Hitt R, et al: Open-label, uncontrolled, multicenter phase II study to evaluate the efficacy and toxicity of cetuximab as a single agent in patients with recurrent and/or metastatic squamous cell carcinoma of the head and neck who failed to respond to platinum-based therapy. J Clin Oncol 25:2171-7, 2007
18. Vermorken J B, Mesia R, Rivera F, et al: Platinum-based chemotherapy plus cetuximab in head and neck cancer. N Engl J Med 359:1116-27, 2008
19. Terme M, Pernot S, Marcheteau E, et al: VEGFA-VEGFR pathway blockade inhibits tumor-induced regulatory T-cell proliferation in colorectal cancer. Cancer Res 73:539-49, 2013
20. Doedens A L, Phan A T, Stradner M H, et al: Hypoxia-inducible factors enhance the effector responses of CD8(+) T cells to persistent antigen. Nat Immunol 14:1173-82, 2013
21. de Almeida P E, Mak J, Hernandez G, et al: Anti-VEGF Treatment Enhances CD8(+) T-cell Antitumor Activity by Amplifying Hypoxia. Cancer Immunol Res, 2020
22. Seiwert T Y, Burtness B, Mehra R, et al: Safety and clinical activity of pembrolizumab for treatment of recurrent or metastatic squamous cell carcinoma of the head and neck (KEYNOTE-012): an open-label, multicentre, phase 1b trial. Lancet Oncol 17:956-965, 2016
23. Ferris R L, Blumenschein G, Jr., Fayette J, et al: Nivolumab for Recurrent Squamous-Cell Carcinoma of the Head and Neck. N Engl J Med 375:1856-1867, 2016
24. Taylor M H R D, Brose M S, et al.: A phase 1b/2 trial of lenvatinib plus pembrolizumab in patients with squamous cell carcinoma of the head and neck. J Clin Oncol 36:6016, 2018
25. Wolchok J D, Hoos A, O'Day S, et al: Guidelines for the evaluation of immune therapy activity in solid tumors: immune-related response criteria. Clin Cancer Res 15:7412-20, 2009
26. Karar J, Maity A: PI3K/AKT/mTOR Pathway in Angiogenesis. Front Mol Neurosci 4:51, 2011
27. Manyam B V, Garsa A A, Chin R I, et al: A multi-institutional comparison of outcomes of immunosuppressed and immunocompetent patients treated with surgery and radiation therapy for cutaneous squamous cell carcinoma of the head and neck. Cancer 123:2054-2060, 2017
28. Venables Z C, Autier P, Nijsten T, et al: Nationwide Incidence of Metastatic Cutaneous Squamous Cell Carcinoma in England. JAMA Dermatol 155:298-306, 2019

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating a cancer in a subject, which method comprises:
(a) determining the presence of a mutation in one or more genes involved in the phosphoinositide 3-kinase (PI3K) signaling pathway in a sample obtained from the subject; and
(b) administering a dose of axitinib to the subject, whereby the cancer in the subject is treated.

2. The method of claim 1, wherein the cancer is unresectable recurrent or metastatic head and neck squamous cell carcinoma (R/M HNSCC).

3. The method of claim 1 or claim 2, wherein the one or more genes involved in the PI3K signaling pathway are selected from PTEN, PIK3CA, and AKT.

4. The method of any one of claims 1-3, wherein the subject has received at least one cancer treatment prior to the administration of the dose of axitinib.

5. The method of claim 5, wherein the at least one cancer treatment is a platinum-based chemotherapeutic agent or a PD-1 inhibitor.

6. The method of any one of claims 1-5, wherein the sample comprises blood, tumor tissue or suspected tumor tissue, lymph node tissue, urine, or saliva.

7. The method of any one of claims 1-6, which further comprises administering a cancer immunotherapeutic to the subject simultaneously with or subsequently to administration of the dose of axitinib.

8. The method of claim 7, wherein the cancer immunotherapeutic is selected from immune checkpoint inhibitors, monoclonal antibodies, cancer vaccines, immune system modulators, and T-cell transfer therapy.

9. The method of claim 7, wherein the cancer immunotherapeutic is an immune checkpoint inhibitor.

10. The method of any one of claims 1-8, which comprises administering to the subject a dose of axitinib of at least 5 mg twice daily.

11. The method of any one of claims 1-10, wherein the subject is a human.

12. Axitinib for use in a method of treating a subject with cancer, wherein the method comprises:
(a) determining whether a test sample from the subject comprises a mutation in one or more genes involved in the phosphoinositide 3-kinase (PI3K) signaling pathway; and
(b) if the test sample from the subject comprises a mutation in one or more genes involved in the PI3K signaling pathway, administering to the subject an effective amount of axitinib.
